(19) United States
(12) Patent Application Publication
(10) Pub. No.: US 2004/0158317 A1
(43) Pub. Date: Aug. 12, 2004

(54) Coated stent with ultrasound therapy

(75) Inventors: Axel F. Brisken, Fremont, CA (US); Robert Zuk, Atherton, CA (US); John McKenzie, San Carlos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pharmasonics, Inc., Sunnyvale, CA (US)

(21) Appl. No.: 10/765,643

(22) Filed: Jan. 26, 2004

Related U.S. Application Data
(63) Continuation of application No. 09/908,487, filed on Jul. 17, 2001.
(60) Provisional application No. 60/218,918, filed on Jul. 18, 2000.

(51) Int. Cl. A61F 2/06

(52) U.S. Cl. 623/1.42; 604/22; 600/439

(57) ABSTRACT

Methods and apparatus for inhibiting hyperplasia following stent placement are provided. The stents will be coated with pharmaceutical agent(s) and the methods provide for directing vibrational energy at the implanted stents or the blood vessel walls or region of implantation. The vibrational energy can have one or more of several beneficial effects. The vibrational energy may directly inhibit hyperplasia, may effect or modulate release of agent from the stent, or may enhance permeability of the blood vessel wall.

### CROSS-REFERENCES TO RELATED APPLICATIONS

This application is a continuation of U.S. application Ser. No. 09/908,487 (Attorney Docket No. 017148-003610US), filed Jul. 17, 2001, which application claimed the benefit under 35 USC 119(e) of provisional Application No. 60/218,918 (Attorney Docket No. 017148-003600US), filed on Jul. 17, 2000, the full disclosures which are incorporated herein by reference.

### BACKGROUND OF THE INVENTION

1. Field of the Invention

The present invention relates generally to medical methods, apparatus, and kits. More particularly, the present invention relates to methods, apparatus, and kits for treating vascular sites to inhibit hyperplasia.

A number of percutaneous intravascular procedures have been developed for treating atherosclerotic disease in a patient's vasculature. The most successful of these treatments is percutaneous transluminal angioplasty (PTA) which employs a catheter having an expansible distal end, usually in the form of an inflatable balloon, to dilate a stenotic region in the vasculature to restore adequate blood flow beyond the stenosis. Other procedures for opening stenotic regions include directional arthrectomy, rotational arthrectomy, laser angioplasty, stents and the like. While these procedures, particularly PTA followed by stenting, have gained wide acceptance, they continue to suffer from the subsequent occurrence of restenosis.

Restenosis refers to the re-narrowing of an artery within weeks or months following an initially successful angioplasty or other primary treatment. Restenosis afflicts up to 50% of all angioplasty patients and results at least in part from vascular smooth muscle cell proliferation in response to the injury caused by the primary treatment, generally referred to as “neointimal hyperplasia.” Blood vessels in which significant restenosis occurs will require further treatment.

A number of strategies have been proposed to reduce restenosis. Such strategies include prolonged balloon inflation, treatment of the blood vessel with a heated balloon, treatment of the blood vessel with ionizing radiation, the administration of anti-thrombotic drugs following the primary treatment, stenting of the region following the primary treatment, and the like. While enjoying different levels of success, no one of these procedures has proven to be entirely successful in treating all occurrences of restenosis.

Recently, there has been significant interest in reducing restenosis in implanted stents by coating stents with drugs or other pharmaceutical agents which are intended to inhibit hyperplasia or have other beneficial biological effects. A number of drugs have been proposed, including anti-coagulants, anti-proliferative agents, anti-inflammatory agents, antibiotics, antioxidants, and the like. While holding great promise, the use of drug-coated stents still suffers from certain shortcomings. For example, the need to provide a high initial dose or bolus of the drug can expose the patient to potentially toxic levels of at least some of the drugs. Moreover, once a drug-coated stent has been implanted, there is little ability to depart from whatever drug release profile has been programmed into that stent prior to implantation. Thus, there is little ability to address unexpected occurrences.

An alternative approach for inhibiting hyperplasia is described in U.S. Pat. No. 6,210,393, assigned to the assignee of the present application. This patent discloses methods for inhibiting hyperplasia within implanted stents and at other sites of vascular intervention by exposing those sites to vibrational energy under conditions selected to inhibit cell proliferation and neointimal hyperplasia. Vibrational energy is usually applied using a transducer carried by a catheter, typically at the time of stent implantation. Although, the methods are effective, they are intended for treatment only at one or more distinct points in time. Thus, they are unable to provide a continuous, controlled therapy over an extended period of time.

For these reasons, it would be desirable to provide alternative and additional methods, apparatus, and kits for the inhibition of neointimal hyperplasia at vascular treatment sites, particularly in coronary and other arteries following angioplasty, stenting, and other recanalization treatments. It would be particularly desirable to provide methods, apparatus, and kits which could combine the benefits of vibrational therapy, including lung-toxicity, focus treatment, and the like, with the controlled long-term benefits of stent-based drug delivery. More particularly, it would be desirable to utilize the benefits of vibrational therapy to reduce the need to provide an initial bolus of the drug from an implanted drug-coated stent and/or modulate and control the release of drug from a drug-coated stent after the stent has been implanted. Some of these objectives will be met by the invention described hereinafter.

2. Description of the Background Art

Young and Dyson (1990) UMB 16:261-269, describe experiments where daily low doses of ultrasound, typically at approximately 1 MHz and 0.1 mechanical index (MI), significantly enhance the growth of new blood vessels in the skin of adult rats. The applied ultrasound affects the release of angiogenic factors. Asahara et al. (1996) Circulation 94:3291-3303, describe the administration of plasmid DNA expressing DEGF following balloon angioplasty in rabbits. It was found that the DEGF substantially accelerated re-endothelization of the blood vessels.

Numerous patents and patent publications are related to methods and catheters for delivering vibrational energy to blood vessels and other body structures. See, for example, U.S. Pat. Nos. 5,059,166; 5,163,421; 5,199,939; 5,213,561; 5,269,291; 5,302,168; 5,312,430; 5,315,998; 5,318,014; 5,344,395; 5,362,309; 5,474,531; 5,514,086; 5,527,337; 5,599,294; 5,599,844; 5,616,114; 5,836,896; 5,840,031; 5,846,218; 6,210,393; and published PCT Application WO 98/48711.

Pertinent publications include:

Alter et al., “Ultrasound inhibits the adhesion and migration of smooth muscle cells in vitro” Ultrasound in Medicine (1998) 24 (5):711-721.

Alter et al., “Ultrasound inhibits the adhesion and migration of smooth muscle cells in vitro” Ultrasound in Medicine (1998) 24(5):711-721.

Bendeck et al., “Inhibition of matrix metalloproteinase activity inhibits smooth muscle cell migration but not neointimal thickening after arterial injury” (1996) Circ. Res. 78:38-43.

He et al., “Application of ultrasound energy for intracardiac ablation of arrhythmias” Eur. Heart J. (1995) 16:961-966.

Rosenchein et al., “Experimental ultrasonic angioplasty: Disruption of atherosclerotic plaques and thrombi in vitro and arterial recanalization in vitro” JACC (1990) 15:711-717.

Kaufman et al., “Lysis and viability of cultured mammalian cells exposed to 1 MHz ultrasound” Ultrasound Med. Biol. (1977) 3:21-25.

Schwartz et al., “Vascular cell proliferation dynamics: Implications for gene transfer and restenosis” Gene Translation in the Cardiovascular System: Experimental Approaches and Therapeutic Implications (1997) Keith L. Marsh, Editor, Kluwer Academic Publications, Netherlands, pp. 293-305.

Siegel et al., “Ultrasound angioplasty” J. Invasive Cardiol. (1991) 3:135.

The use of stents for drug delivery within the vasculature are described in U.S. Pat. Nos. 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,769,883; 5,735,811; 5,700,286; 5,69,400; 5,649,977; 5,637,113; 5,609,629; 5,591,227; 5,551,954; 5,545,208; 5,500,013; 5,464,450; 5,419,760; 5,411,550; 5,342,348; 5,286,254; and 5,163,952. Biodegradable materials are described in U.S. Pat. Nos. 5,876,452; 5,656,297; 5,543,158; 5,484,584; 4,897,268; 4,883,666; 4,832,686; and 3,976,071. The use of hydrocylosiloxane as a rate limiting barrier for drug delivery is described in U.S. Pat. No. 5,463,010.

The full disclosures of each of the above references are incorporated herein by reference.

### BRIEF SUMMARY OF THE INVENTION

The present invention combines the use of “coated” implantable vascular scaffold structures, including stents, grafts, and other omplantable vascular prostheses, with methods and systems for directing vibrational energy at the implanted structures and/or the vascular sites where the structures have been implanted. The scaffold structures will be coated with a pharmaceutical agent, typically a drug or a gene, which will be released into the vascular wall at the site of implantation before, during and/or after the application of the vibrational energy. The vibrational energy will provide one or more of several beneficial interactions with the coated structure.

First, the vibrational energy can provide and/or enhance anti-hyperplasia at the site of implantation during periods of particular vulnerability. For example, the vibrational energy can be directed at the implanted structure and/or site of implantation during and/or immediately following implantation of the scaffold structure. Alternatively or additionally, vibrational energy can be directed at the site of implantation and the implanted scaffold hours, days, weeks, or even months after the time of implantation. And particularly, there is evidence that smooth muscle cell proliferation increases during the period from one week to one month following implantation of a stent or other vascular scaffold structure. The present invention can be used to externally apply vibrational energy to the site of implantation during this period of increased vulnerability in order to enhance the anti-hyperplastic activity of the pharmaceutical agent which will be released at a controlled, but generally decreasing rate from the time of implantation forward.

Second, vibrational energy can be applied according to methods of the present invention in order to enhance absorption of the drug which is being released from the vascular scaffold structure into the vascular wall. Generally, the frequency, the power (mechanical index), and other characteristics of the vibrational energy intended to enhance absorption may differ from those which are intended to directly inhibit hyperplasia and smooth muscle cell proliferation. Thus, it will be possible to treat the site of implantation using different vibrational energies in order to achieve at least two effects of the present invention, i.e., direct inhibition of smooth muscle cell proliferation and hyperplasia as well as enhanced absorption of the pharmaceutical agent being released from the structure (which will also reduce hyperplasia in a manner characteristic of the pharmaceutical agent).

In a third embodiment, vibrational energy may be directed at the implanted vascular scaffold structure in order to effect release of the pharmaceutical agent from the structure. For example, the scaffold structure may be coated with the pharmaceutical agent which in turn is coated with an impermeable layer which prevents release of the drug. The vibrational energy can be directed at the scaffold structure in order to fracture or otherwise render permeable the impermeable barrier, thus initiating release of the pharmaceutical agent from the scaffold structure. In other instances, it will be possible to direct vibrational energy at the implanted vascular scaffold structure in order to modulate, usually increase, the release of drug from the structure. For example, the coated stent is covered with a barrier layer which degrades in the vascular environment, the application of vibrational energy can promote the rate of degradation or dissolution of the barrier layer. Optionally, the barrier layer can also have the pharmaceutical agent dispersed therein, so that an increase in degradation or dissolution has a direct increase on the amount of agent released into the vascular wall.

Thus, the present invention broadly provides methods for inhibiting hyperplasia at a vascular treatment site. The methods comprise directing vibrational energy at or toward the vascular treatment site, where a scaffold structure has been implanted at the treatment site. The scaffold structure is coated with a pharmaceutical agent which is released into the site over time. The vibrational energy directed at the vascular treatment site and/or scaffold structure can have any of the effects described above. In particular, the vibrational energy can be directed at the vascular treatment site at a frequency and a thermal index which will inhibit an acute phase of hyperplasia, typically which occurs at or near the time of implantation. The vibrational energy can provide direct inhibition of hyperplasia at the time of implantation while the pharmaceutical agent which is released from the scaffold structure will provide a prolonged hyperplasia inhibition, typically over at least a week, usually over at least a month, and frequently over several months or longer.

In a first specific aspect of the methods of the present invention, the vibrational energy will be selected to directly inhibit hyperplasia. Preferable, the vibrational energy will not cause significant cavitation in the wall of the blood vessel, and will cause a temperature rise below 10° C. in the blood vessel wall. The vascular smooth muscle cells which have been exposed to the vibrational energy will remain viable but in a quiescent state in the neointimal layer of the blood vessel wall after exposure to the vibrational energy. Migration of the vascular smooth muscle cells into the neointimal layer will not be substantially inhibited, and viability of the vascular smooth muscle cells in the medial layer of the blood vessel will also not be significantly inhibited. Vibrational energy will have a frequency in the range from 20 kHz to 5 MHz, preferably in the range from 300 kHz to 3 MHz, and more preferably in the range from 500 kHz to 1.5 MHz. The vibrational energy will have an intensity (SPTA) in the range from 0.01 W/cm² to 100 W/cm², preferably in the range from 0.1 W/cm² to 20 W/cm², and more preferably in the range from 0.5 W/cm² to 5 W/cm². Together, the frequency and intensity will be selected to produce a mechanical index at the neointimal wall in the range from 0.1 to 50, preferably from 0.2 to 10, and more preferably from 0.5 to 5. The vibrational energy will preferably be directed against the implantation site with a pulse repetition frequency (PRF) in the range from 10 Hz to 10 kHz, preferably with a duty cycle in the range from 0.1 to 100 percent.

In a second specific aspect of the method, the vibrational energy will be directed at the implantation site and/or implanted scaffold structure at a frequency and intensity which result in a mechanical index selected to promote release of the pharmaceutical agent from the implanted scaffold structure. The vibrational energy may cause fracture of a barrier layer, enhanced permeability of the barrier layer, or direct release of the pharmaceutical agent which would otherwise be adhered or bonded to the scaffold structure. Preferably, the mechanical index will be in the range from 0.1 to 50, more preferably from 0.2 to 10, and most preferably from 0.5 to 5. Suitable frequencies and intensities of the vibrational energy will be in the range from 0.01 W/cm² to 100 W/cm², preferably from 0.1 W/cm² to 20 W/cm²(SPTA), and from 20 hHz to 5 MHz, preferably from 300 kHz to 3 MHz, respectively.

In a third specific embodiment of the method of the present invention, the vibrational energy is directed at the implanted scaffold structure and/or site of implantation in the vascular wall at a mechanical index selected to condition the vascular wall to enhance uptake of the pharmaceutical agent, usually by enhancing permeability of the vascular wall. Typically the mechanical index will be in the range from 0.1 to 50, preferably from 0.2 to 10, and more preferably from 0.5 to 5. The frequency will be typically be in the range from 300 kHz to 3 MHz, and the intensity in the range from 0.1 W/cm² to 20 W/cm².

Methods according to the present invention may comprise directing the vibrational energy at the implanted scaffold structure and/or implantation site in the vascular wall one or more times before, during, or after implantation of the scaffold structure. For example, the vibrational energy may be directed at the vascular treatment site at least once at the time of implantation, typically using a catheter for the intravascular delivery of the vibrational energy. Vibrational energy may then be directed at the same site one or more times after the implantation, typically using an external transducer for the transcutaneous delivery of vibrational energy to the site. Such external vibrational energy can be focused or non-focused, depending on the intensity and mechanical index desired. Typically, focused vibrational energy can achieve a higher intensity.

The methods and systems of the present invention can be used to deliver a wide variety of pharmaceutical agents, generally including both drugs (small molecule and macromolecular drugs) as well as genes, gene fragments, and other biologically active nucleic acids. Particularly, the methods can be used to deliver anti-coagulants, such as heparin, hirudin, and GpIIB/IIIA inhibitors; anti-proliferation agents, such as paclitaxol, and nitric oxide; anti-inflammatory agents, such as dexamethasone, and methylprednisolone; antibiotics such as rapamyacin; anti-oxidants, such as probucol; and the like. Genes and nucleic acids which can be delivered according to the methods of the present invention include genes which express vascular endothelial growth factor (VEGF), thymidine kinase, eNOS; antisense oligonucleotides, such as c-myc.; and the like. The pharmaceutical agents can be coated, layered, painted, bonded, or otherwise coupled or attached onto the scaffold structure by the methods taught in the art, such as in the patents which have been incorporated by reference above. In addition or as an alternative to directly coating the scaffold structure, the pharmaceutical agent may be dispersed in a biodegradable matrix which is coated or otherwise layered on the surface of the scaffold structure. Such biodegradable matrices may be comprise polylactic acid, polyglycolic acid, or the like.

The present invention further comprises implantable scaffold structures, wherein the structures are coated or otherwise covered with a pharmaceutical agent which is adapted for release into an adjacent vascular region when vibrational energy is applied to the structure. The pharmaceutical agent may comprise any of the drugs, genes, nucleic acids, or the like, described above, and the agents will typically be formulated in a biodegradable matrix, where biodegradation or dissolution of the matrix is enhanced upon the application of vibrational energy. For example, the biodegradable matrix may comprise polylactic acid and/or polyglycolic acid. In a still further aspect of the present invention, kits comprise catheters having vibrational transducers and instructions for use according to the methods of the present invention. In particular, the instructions will set forth methods for using the catheters to apply vibrational energy to coated stents in order to effect, enhance, or otherwise modulate the release and/or absorption of pharmaceutical agents from the stents or other vascular scaffold structures into a blood vessel wall.

Kits may also comprise external vibrational transducers together with instructions for use to externally apply vibrational energy to effect or modulate the release of pharmaceutical agents from implanted vascular scaffold structures. The kits of the present invention may further comprise package materials, such as a box, pouch, tray, or the like, wherein at least some of the components may be provided and maintained in a sterile condition. The kits may comprise other components selected to facilitate practice of the methods of the present invention.

### BRIEF DESCRIPTION OF THE DRAWINGS

FIG. 1 illustrates a first exemplary treatment method according to the present invention where a catheter having a vibrational transducer is used to direct vibrational energy to an implanted vascular scaffold structure within a blood vessel.

FIG. 2 illustrates a second exemplary treatment method according to the present invention where an external vibrational transducer is used to deliver vibrational energy to an implanted vascular scaffold structure within a coronary artery.

FIG. 3 is a cross-sectional view of a blood vessel having an implanted vascular scaffold structure being treated with a vibrational catheter according to the methods of the present invention.

FIG. 4 is a detailed view taken along line 4-4 of FIG. 3.

FIGS. 5A-5D illustrate different coated vascular scaffold structures which can be utilized in the methods of the present invention.

FIGS. 6A and 6B illustrate a particular method for using vibrational energy to degrade a pharmaceutical agent-containing layer formed on a scaffold structure according to the present invention.

FIG. 7 illustrates a kit according to the present invention comprising a vibrational catheter and instructions for its use.

FIG. 8 illustrates a second kit according to the present invention comprising an external vibrational transducer and instructions for its use performing the methods of the present invention.

### DESCRIPTION OF THE SPECIFIC EMBODIMENTS

Blood vessels, particularly coronary arteries, which have been treated by angioplasty and stent implantation are subject to reocclusion due to hyperplasia and other injury responses, as discussed above. The present invention is directed at improved vascular scaffold structures which are coated with pharmaceutical agents and methods for treating such improved scaffold structures before, during, or after implantation by exposing the structures to vibrational energy intended to interact with the structure and/or the implantation site within the vascular wall in order to enhance prevention of reocclusion. In particular, the present invention provides for the direction of vibrational energy at the implanted vascular scaffold structure and/or the blood vessel wall at the site of implantation in order to achieve at least one of several possible beneficial effects, as discussed above.

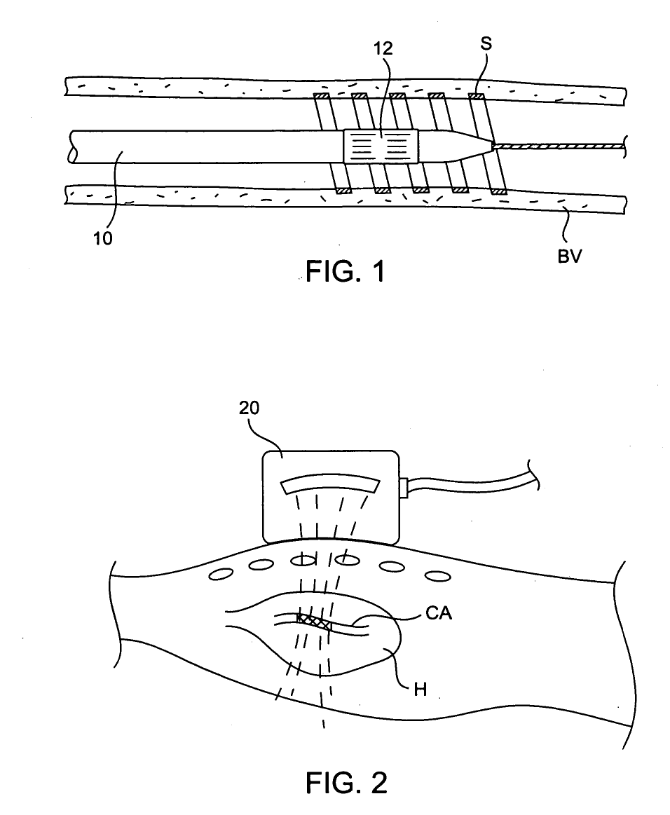

Referring now to FIGS. 1 and 2, vibrational energy may be applied to the patient in either of two different ways. In FIG. 1, an intravascular catheter 10 having a vibrational transducer 12 is intravascularly introduced into a blood vessel BV so that the transducer lies within a region of the blood vessel where a vascular scaffold structure or stent S has been implanted. The stent will be coated with a pharmaceutical agent which is to be released into the wall of the blood vessel BV at the site of the implantation. The pharmaceutical agent can be any of the agents discussed previously, or could be any of the agents identified in the patents related to coated stents which have previously been incorporated herein by reference. The vibrational transducer 12 will be energized to vibrate and emit vibrational energy, typically ultrasonic energy, that travels radially outwardly so that it engages and interacts with the structure of the stent and/or the blood vessel wall, depending on the particular biological effect which is desired. A wide variety of intravascular catheters 10 may be used, including those described in the prior U.S. Patents and pending patent applications which have been incorporated herein by reference above.

The use of an intravascular catheter to direct vibrational energy to the site of implantation within the blood vessel will generally be preferred at the time of initial implantation of the vascular scaffold structure. Conveniently, the vibrational transducer 12 could be provided on a balloon or other scaffold structure delivery catheter so that the vibrational energy could be delivered during and/or immediately after scaffold implantation.

In some instances, however, it will be desirable to deliver the vibrational energy to the region of the scaffold structure at a time days, weeks, or even months after initial implantation of the structure. In such cases, it will usually be desirable to use an external vibrational transducer 20 such as the transducer described in co-pending application 09,255,290, the full disclosure which is incorporated herein by reference. The external vibrational transducer 20 may deliver focused or unfocused ultrasonic or other vibrational energy transcutaneously, typically through the patient's chest and ribs into the coronary artery CA of the heart H. By properly choosing the characteristics of vibrational energy, hyperplasia can be directly inhibited, the release of drug from the scaffold structure can be modulated and/or the permeability of the blood vessel wall can be enhanced, even at times well after implantation of the scaffold structure.

Mechanical index and duration of the treatment are the most important treatment parameters. The mechanical index (MI) is a function of both the intensity and the frequency of the vibrational energy produced, and is defined as the peak rarefactional pressure (P) expressed in megaPascals divided by the square root of frequency (f) expressed in megaHertz:

$$MI = \frac{P}{\sqrt{f}}$$

The duration of treatment is defined as the actual time during which vibrational energy is being applied to the arterial wall. Duration will thus be a function of the total elapsed treatment time, i.e., the difference in seconds between the initiation and termination of treatment; burst length, i.e., the length of time for a single burst of vibrational energy; and pulse repetition frequency (PRF). Usually, the vibrational energy will be applied in short bursts of high intensity (power) interspersed in relatively long periods of no excitation or energy output. An advantage of the spacing of short energy bursts is that heat may be dissipated and operating temperature reduced.

Broad, preferred, and exemplary values for each of these parameters for direct inhibition of hyperplasia is set forth in the following table.

PREFERRED AND EXEMPLARY
TREATMENT CONDITIONS

| | BROAD | PREFERRED | EXEMPLARY |
|---|---|---|---|
| Mechanical Index (MI) | 0.1 to 50 | 0.2 to 10 | 0.5 to 5 |
| Intensity | 0.01 to 100 | 0.1 to 20 | 0.5 to 5 |
| (SPTA, W/cm²) | | | |
| Frequency (kHz) | 20 to 5000 | 300 to 3000 | 500 to 1500 |
| Elapsed Time (sec.) | 10 to 900 | 30 to 500 | 60 to 300 |
| Duty Cycle (%) | 0.1 to 100 | 0.2 to 10 | 0.2 to 2 |
| Pulse Repetition | 10 to 10,000 | 100 to 5000 | 300 to 3000 |
| Frequency (PRF)(Hz) | | | |

The above values are intended for treatments where the primary objective is to directly inhibit hyperplasia based on the biological effect of the vibrational energy on the blood vessel wall. Such treatments will generally be performed as a supplement to the treatment effected by the pharmaceutical agent. For example, it is desired to provide enhanced hyperplasia inhibition at the time of scaffold structure implantation, vibrational energy can be applied using an intravascular catheter 10 as illustrated in FIG. 1. In this way, it will be possible to reduce the amount of pharmaceutical agent to be released from the stent. Viewed another way, the pharmaceutical agent which is released from the stent over time will provide a prolonged or chronic inhibition of hyperplasia. Alternatively, it is believed that an extra measure of hyperplasia inhibition is required at some period after implantation. For example from one week to one month, then an external vibrational transducer 20, as illustrated in FIG. 2, can be used. Again, the dosage of pharmaceutical agent can be released to lessen the risk of toxic or other adverse effects since the vibrational energy is providing an independent source of hyperplasia inhibition.

The characteristics of the vibrational energy used to control the release of pharmaceutical agent from the scaffold structure and/or to modulate the permeability of the blood vessel wall to the released pharmaceutical agent may vary widely, depending on the particular therapy being performed.

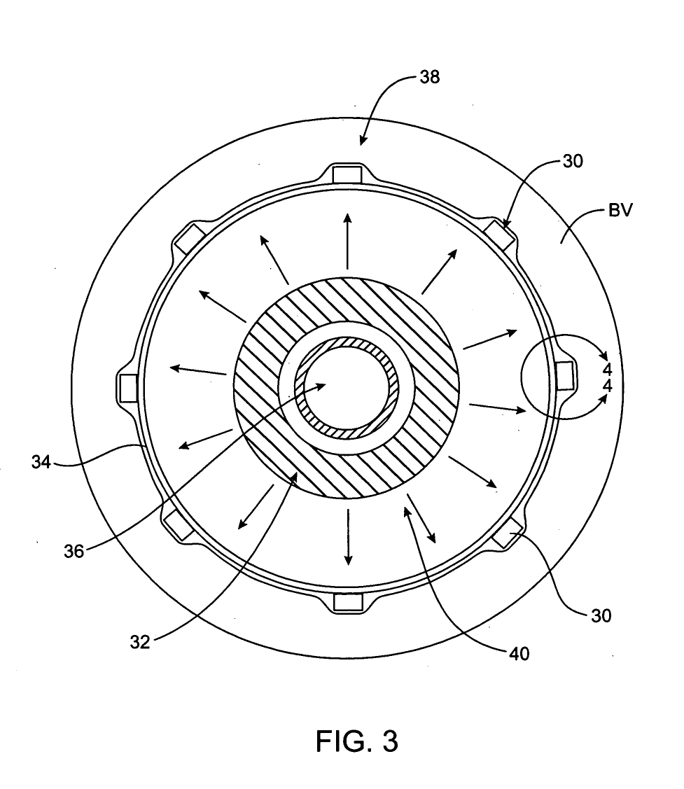

Referring now to FIG. 3, a blood vessel having an implanted scaffold structure comprising struts 30 which are coated with pharmaceutical agents, described in more detailed below, is treated with a vascular catheter 32 including both a vibrational transducer and an expansible balloon 34. The catheter will typically include a guide wire lumen 36, and a vibrational transducer will emit vibrational energy, typically ultrasonic energy, radially outwardly as indicated by the arrows emanating from the surface of the catheter 32. Conveniently, the balloon 34 can be used for deploying the vascular scaffold structure, and the vibrational energy can be directed into the struts 30 of the structure as well as the blood vessel wall 38 during and for a period following the scaffold deployment. The balloon can be inflated with an inflation medium 40 which is selected to transmit the vibrational energy in an efficient manner.

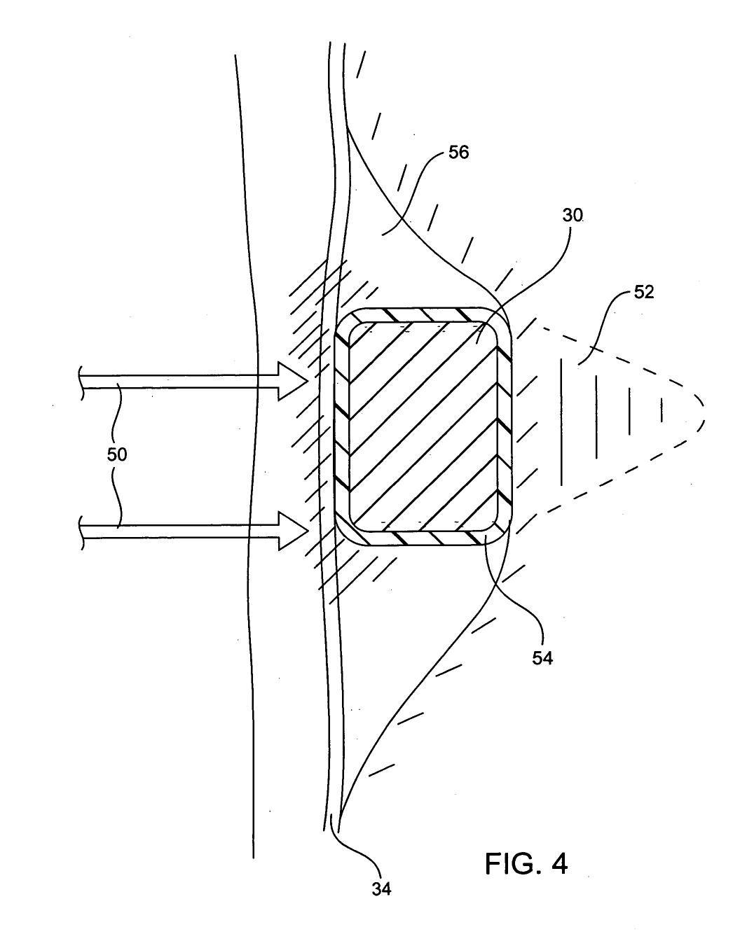

Referring now to FIG. 4, the local effect of the incident vibrational energy represented by arrows 50 in a region of scaffold implantation will be described. The vibrational energy 50 passes through the balloon 34 and into the stent strut 30. There will usually be a vibrational shadow zone 52 on the other side of the strut 30 within the vascular wall. The vibrational energy 50 may have any of the effects described above. For example, the vibrational energy may directly inhibit hyperplasia within the vascular wall. Alternatively, the vibrational energy 50 may modulate a release of pharmaceutical agent from the pharmaceutical agent layer 54, releasing the agent into the vascular wall as well as into an entrapment zone 56 defined by the strut 30, the vascular wall, and the surface of balloon 34. As a third alternative, the vibrational energy may cause the stent structure to vibrate in such a way that the vascular wall in the region 52 is caused to become more permeable to the pharmaceutical agent which is being released.

In a preferred aspect of the present invention, the incident illuminating beam from the transducer operates in the 2 to 4 MI (Mechanical Index) range. As such, these short range higher amplitude (perhaps in the 4 to 8 MI range) standing and shear waves have a greater ability to temporarily permeabilize cell membranes for higher rates of transfection (for example: in the case of the plasmid DNA applied, i.e., “coated” onto the stent struts) or a greater therapeutic effect (for example: in the case of an uncoated stent).

On the lateral wall of the stent struts, multiple internal reflections of each waveform cycle will also result in a minor contribution, but a small amount of shear may be present due to the Poisson's ratio of the material. The emission from the back surface of the stent will preferably be phase shifted with respect to the bypassing incident wave. Furthermore, the emission from the back surface may also act in a diffractive manner, with the amplitude rapidly decaying in both the forward and lateral directions. Due to the reduced amplitude of the transmitted wave, the magnitude of the shear will be substantially less. A few wavelengths behind the stent strut, the incident beam will have closed again, with virtually no acoustic evidence for the presence of the stent.

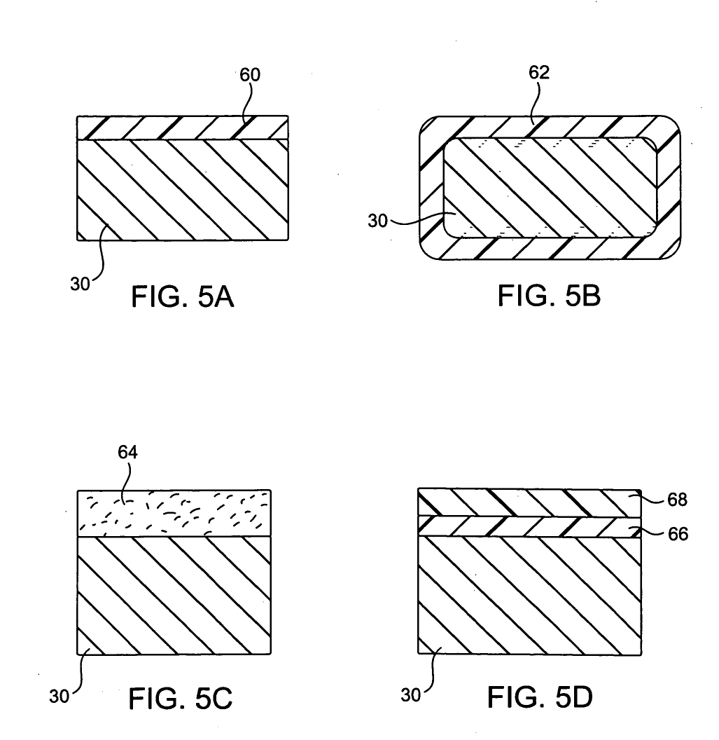

Referring now to FIGS. 5A-5D, the vascular scaffold structure may be coated with the pharmaceutical agent in a variety of ways. For example, a strut 30 may be coated with a layer 60 consisting essentially of the pharmaceutical agent or the agent and a non-active carrier, on a single surface thereof. Usually, this surface will be intended to contact the wall of the blood vessel so that the layer 60 is embedded into the blood vessel wall. Alternatively, the strut 30 can be coated with a layer 62 on all sides, as illustrated in 5B. As a still further alternative, a strut 30 may be coated with a layer 64 which comprises the drug dispersed in a biodegradable matrix. The biodegradable matrix, which may comprise polylactic acid and/or polyglycolic acid, will be formulated to degrade in a vascular environment. Optionally, the layer can be formulated so that degradation can be enhanced by the application of vibrational energy, thus allowing modulation of the release over time using a vibrational transducer, usually an external transducer as illustrated in FIG. 2. As a still further alternative, a strut 30 may be coated with a pharmaceutical agent-containing layer 66 which is covered by a permeable or non-permeable barrier layer 68. The barrier layer 68 can be permeable or porous in order to provide for controlled release of drug from the drug layer 66. Alternatively, the barrier layer can be impermeable, where release is effected by the application of vibrational energy to fracture or otherwise permit release of the drug through the barrier. A wide variety of other pharmaceutical agent coating techniques are known in the art, as described in the prior patent publications, which have been incorporated herein by reference.

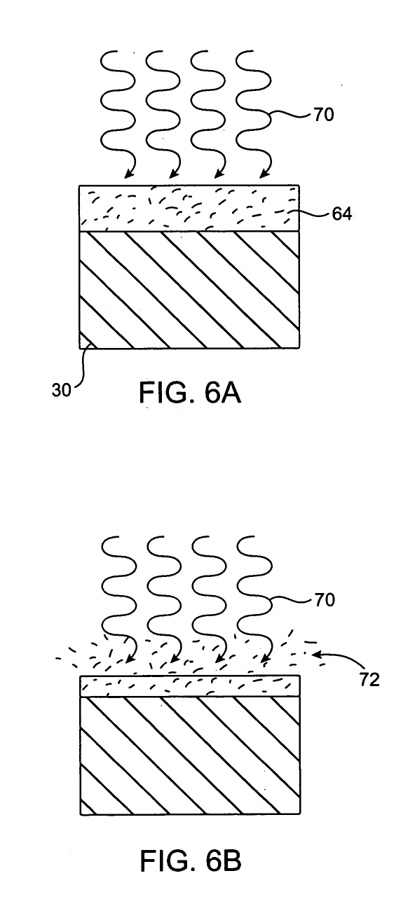

Referring now to FIG. 6A and 6B, a method which relies on the application of vibrational energy 70, (schematically illustrated using arrows) will be described. The vibrational energy 70 contacts the dispersed pharmaceutical agent layer 64, causing a matrix in the layer to degrade or dissolve over time. The layer will dissolve, as shown in FIG. 6B, releasing drug particles 72 into the blood vessel walls and/or an entrapment zone as illustrated in FIG. 4.

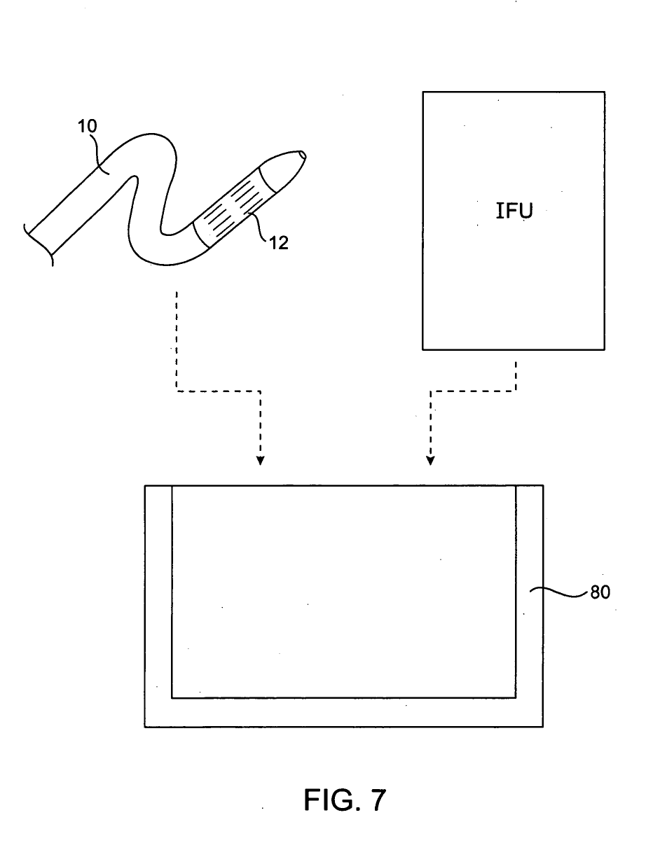

Referring now to FIG. 7, the kit according to the present invention includes at least one catheter 10 having a vibrational transducer 12 for applying vibrational energy according to the methods described above. The kit further comprises instructions for use IFU setting forth these methods, the kit will typically further comprise a package 80, typically a box, tube, tray, pouch, or the like. The catheter 10 may be maintained sterilely within the package 80, and the kit may comprise a variety of other components intended to facilitate performance of the methods of the present invention.

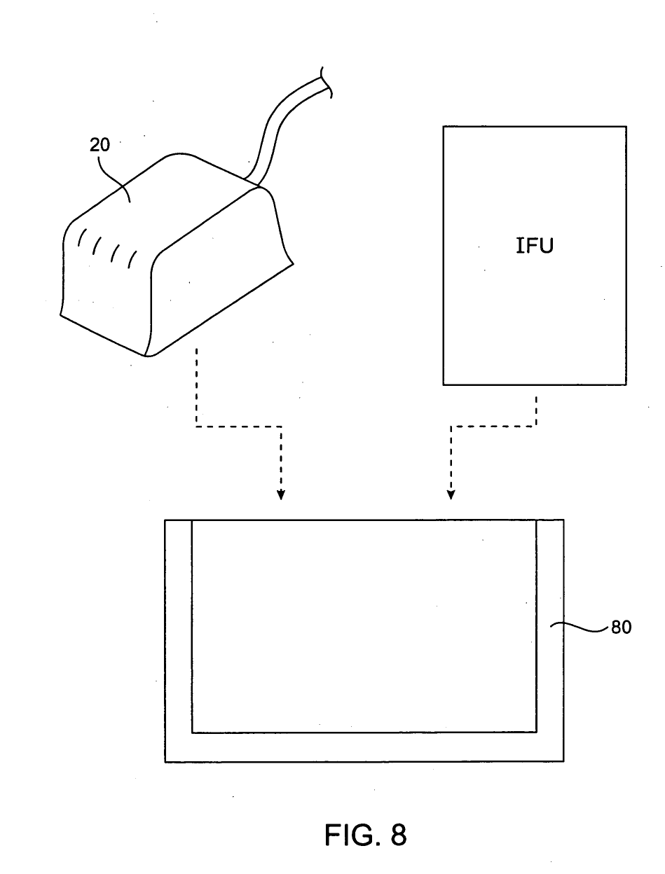

Referring now to FIG. 8, a second kit comprising an external transducer 20 and instructions for use IFU for performing the external treatment protocols according to the methods of the present invention. The kit will also comprise a container 80, and may further comprise other components intended to facilitate performance of the methods of the present invention.

While the above is a complete description of the specific embodiments of the invention, variations, alternatives, modifications, and equivalence may be used and provided. Therefore, the above description should not be taken as limiting the scope of the invention which is defined by the appended claims.

What is claimed is:

1. A method for inhibiting hyperplasia at a vascular treatment site, said method comprising:
directing vibrational energy at the vascular treatment site, wherein a scaffold structure has been implanted at said site, said scaffold structure being coated with a pharmaceutical agent which is released into the site over time, wherein directing vibrational energy comprises positioning a transducer on a catheter at the vascular treatment site and driving the transducer to emit the vibrational energy at the same time as the scaffold structure is implanted.

2. A method as in claim 1, wherein the vibrational energy is directed at the site at the time of implantation of the scaffold structure at a frequency and thermal index which will inhibit an acute phase of the hyperplasia, wherein the pharmaceutical agent is released over a period of at least one week following implantation to provide a longer term inhibition.

3. A method as in claim 2, wherein the vibrational energy does not cause significant cavitation in a wall of the blood vessel.

4. A method as in claim 2, wherein the vibrational energy causes a temperature rise below 10° C. in the wall of the blood vessel.

5. A method as in claim 2, wherein vascular smooth muscle cells at least mostly remain viable but in a quiescent state in the neointimal layer after exposure to the vibrational energy.

6. A method as in claim 2, wherein migration of vascular smooth muscle cells into the neointimal layer is not substantially inhibited.

7. A method as in claim 2, wherein viability of vascular smooth muscle cells in a medial layer of the blood vessel is not significantly inhibited.

8. A method as in claim 2, wherein the vibrational energy has a frequency in the range from 20 kHz to 5MHz.

9. A method as in claim 8, wherein the intensity is in the range from 0.01 W/cm² to 100 W/cm².

10. A method as in claim 9, wherein the frequency and intensity are selected to produce a mechanical index at the neointimal wall in the range from 0.1 to 50.

11. A method as in claim 2, wherein the vibrational energy is directed against the implantation site with a pulse repetition frequency (PRF) in the range from 10 Hz to 10 kHz.

12. A method as in claim 2, wherein the energy is directed against the implantation site with a duty cycle in the range from 0.1 to 100 percent.

13. A method as in claim 1, wherein the vibrational energy is directed at a mechanical index selected to effect or promote release of the pharmaceutical agent from the implanted scaffold structure.

14. A method as in claim 13, wherein the frequency is in the range from 20 kHz to 5 MHz and the intensity is in the range from 0.01 w/cm² to 100 W/cm².

15. A method as in claim 1, wherein the vibrational energy is directed at a mechanical index selected to condition the vascular wall to enhance uptake of the pharmaceutical agent.

16. A method as in claim 15, wherein the frequency is in the range from 300 kHz to 3 MHz and the intensity is in the range from 0.1 w/cm² to 20 W/cm².

17. A method as in claim 1, further comprising directing vibrational energy at the vascular treatment site at least one additional time.

18. A method as in claim 17, wherein vibrational energy is directed at the vascular treatment site at least once at the time of implanting the scaffold structure and at least once one day or longer following implantation.

19. A method as in claim 1, wherein directing vibrational energy comprises externally generating vibrational energy and directing the vibrational energy transcutaneously to the vascular treatment site.

20. A method as in claim 19, wherein externally generating the vibrational energy comprises focusing an externally generated acoustic beam at the vascular treatment site.

21. A method as in claim 1, wherein the pharmaceutical agent comprises an agent selected from the group consisting of:
anti-coagulants (heparin, hirudin, GpIIB/IIIA inhibitors), anti-proliferation agents (paclitaxol, nitric oxide), anti-inflammatory agents (dexamethasone, methylprednisolone), antibiotics (rapamyacin) and anti-oxidants (probucol).

22. A method as in claim 1, wherein the pharmaceutical agent comprises a nucleic acid sequence.

23. A method as in claim 22, wherein the nucleic acid sequence comprises genes expressing VEGF, thymidine kinase, eNOS and antisense oligonucleotides such as c-myc.

24. A method as in claim 1, wherein the pharmaceutical agent is directly layered onto the scaffold structure.

25. A method as in claim 1, wherein the pharmaceutical agent is dispersed in a biodegradable matrix applied to the surface of the scaffold structure.

26. A method as in claim 25, wherein the biodegradable matrix comprises polylactic acid or polyglycolic acid.
